(19) United States
(12) Patent Application Publication
(10) Pub. No.: US 2019/0351175 A1
(43) Pub. Date: Nov. 21, 2019

(54) OXYGEN CONCENTRATOR

(71) Applicant: NGK SPARK PLUG CO., LTD., Nagoya-shi (JP)

(72) Inventors: Masanori SUDA, Nagoya-shi (JP); Taku HIRANO, Nagoya-shi (JP); Keiko NII, Nagoya-shi (JP)

(73) Assignee: NGK SPARK PLUG CO., LTD., Nagoya-shi (JP)

(21) Appl. No.: 16/410,410

(22) Filed: May 13, 2019

(30) Foreign Application Priority Data
May 15, 2018 (JP) 2018-093536

(51) Int. Cl. A61M 16/10; B01D 53/047

(52) U.S. Cl. CPC A61M 16/101; B01D 53/047; A61M 2202/0208; B01D 2257/102; A61M 2205/502; B01D 2256/12

(57) ABSTRACT

In an oxygen concentrator, a housing has a through hole formed therein, and a handle portion provided adjacent to the through hole. The through hole is formed so as to extend toward an other side in an axial direction of the housing from a first opening, the first opening being defined at least by a back-side end of a top face portion, two extension portions, and an upper end of the handle portion, and has an interior space continuously extending between the first opening and a second opening defined at least by a lower end of the handle portion and an upper end of the back face main body portion and disposed on the other side in the axial direction with respect to the first opening.

### BACKGROUND OF THE INVENTION

### 1. Field of the Invention

The present invention relates to an oxygen concentrator.

### 2. Description of the Related Art

In recent years, oxygen inhalation therapy has been known as one of the most effective therapies for respiratory diseases. In oxygen inhalation therapy, a patient is provided with oxygen gas or oxygen-concentrated gas. Recently, an oxygen concentrator for directly separating oxygen-concentrated gas from air has been developed as an oxygen supply source.

For utilization in various circumstances by a patient, a portable oxygen concentrator of this type has been proposed. For example, Patent Document 1 discloses a portable oxygen concentrator having rechargeable batteries for operating electronic devices.

[Patent Document 1] Japanese Patent Application Laid-Open (kokai) No. 2007-167653

### 3. Problems to be Solved by the Invention

A portable oxygen concentrator desirably has a holding part (handle) at a position convenient for holding, since a user often holds it for moving or operation. In this connection, for example, the oxygen concentrator disclosed in Patent Document 1 has a carrying handle at the top of a housing in a laterally bridging manner.

However, the oxygen concentrator of Patent Document 1 necessarily has a large height. This is because its housing has a horizontally extending opening formed on the upper side of a display panel (user interface panel) and provided on a front face portion thereof, while a holding part (carrying handle) extends over the opening in a bridging manner. Specifically, by providing a horizontally extending opening, the height of the housing increases by the vertical width of the opening. As a result of providing the holding part (carrying handle) over the opening, the height of the housing further increases by the vertical width of the holding part in addition to the vertical width of the opening. Moreover, according to the structure disclosed in Patent Document 1. in order to ensure the strength of the holding part (carrying handle), the holding part must have an increased size in the vertical and depth directions to a certain extent. Accordingly, the housing becomes bulky.

### SUMMARY OF THE INVENTION

The present invention has been made to address the above problems, and an object thereof is to provide an oxygen concentrator having a handle portion whose strength is ensured while suppressing an increase in the overall vertical size of the oxygen concentrator.

The above object has been achieved by providing (1) an oxygen concentrator which comprises a housing accommodating a plurality of components and extending in an axial direction. The housing comprises an outer circumferential surface portion having a front face portion extending along the axial direction, a back face portion disposed opposite the front face portion, and a pair of side face portions disposed between the front face portion and the back face portion in a mutually facing manner, a top face portion facing toward one side in the axial direction, and a bottom face portion facing toward the other side in the axial direction. The top face portion is disposed so as to be inclined toward the one side in the axial direction in a direction from the front face portion toward the back face portion and such that an end of the top face portion located toward the back face portion is offset from the back face portion toward the front face portion. An end portion of each of the two side face portions, which end portion is located on the one side in the axial direction, has an inclined portion extending from the front face portion toward the back face portion along the top face portion, and an extension portion extending toward the back face portion further than the top face portion. The back face portion has a handle portion extending between the extension portions of the two side face portions in a bridging manner, and a back face main portion facing the handle portion in the axial direction with a gap intervening therebetween. A first opening is defined at least by the end of the top face portion on the side toward the back face portion, the two extension portions and the handle portion. A second opening is defined at least by an end of the handle portion on the other side in the axial direction and an end of the back face main portion on the one side in the axial direction and is disposed on the other side in the axial direction with respect to the first opening. A through hole is formed which extends from the first opening toward the other side in the axial direction and has an interior space continuously extending between the first opening and the second opening.

In the above oxygen concentrator, portions (extension portions) of the two side face portions are disposed so as to extend toward the back face portion side (hereinafter also referred to as the back side) so as to utilize a space located backward of the back-side end of the top face portion, and a portion (handle portion) of the back face portion is connected to the two extension portions. By virtue of such a structure, a user can hold the portion of the back face portion as a handle. Further, since the handle portion, and the two extension portions connected to the opposite sides of the handle portion are less likely to cause an increase in axial dimension, an increase in the overall size of the oxygen concentrator in the axial direction can be suppressed. That is, in the form of use of the oxygen concentrator with the one side in the axial direction corresponding to the upper side thereof and the other side in the axial direction corresponding to the lower side thereof, an increase in the overall vertical size thereof can be suppressed.

Further, the through hole adjacent to the handle portion is formed such that the through hole extends downward from the first opening (an opening defined at least by the back-side end of the top face portion, the two extension portions and the handle portion) and has an interior space continuously extending between the first opening and the second opening (an opening defined at least by the end (on the other side in the axial direction) of the handle portion and the end (on the one side in the axial direction) of the back face main body portion and disposed on the other side of the first opening in the axial direction). Thus, the interior space of the through hole can be utilized as a space for allowing the user to insert his/her hand therein for holding the handle portion. Further, since the through hole extends from the first opening toward the other side in the axial direction and is adjacent to the handle portion, despite the handle portion having a large axial thickness on account of the large depth of the through hole, the overall size of the oxygen concentrator in the axial direction; i.e., the overall vertical size of the oxygen concentrator, is not prone to increase. Therefore, the strength of the handle portion can be readily ensured while an increase in the overall vertical size of the oxygen concentrator is suppressed.

In a preferred embodiment (2) of the above oxygen concentrator (1), a maximal width of the handle portion along the axial direction is greater than a maximal width along a direction from the front face portion to the back face portion.

By employing such a dimensional feature, since the handle portion can assume a greater axial width, when the user lifts the oxygen concentrator while holding the handle portion, deflection of the handle portion toward the one side in the axial direction is readily restrained. In this manner, the strength of the handle portion is further ensured. Further, despite such a relative increase in the maximal width of the handle portion in the axial direction, the overall size of the oxygen concentrator in the axial direction (i.e., the overall vertical size of the oxygen concentrator) is not prone to increase.

In another preferred embodiment (3) of the oxygen concentrator (1) or (2), a display is provided on the top face portion. This implements an oxygen concentrator allowing easy visual recognition of the display from above. Further, since the display can be provided so as to be exposed from the inclined top face portion, a space under the inclined face portion (top face portion), which encounters difficulty in disposing large components, can be effectively utilized for disposing display-related components (display panel, circuit board, wiring, etc.).

### Effect of the Invention

The oxygen concentrator of the present invention can have a handle portion whose strength is ensured while an increase in the vertical size of the oxygen concentrator is suppressed.

### BRIEF DESCRIPTION OF THE DRAWINGS

FIG. 1 is a front view showing an oxygen concentrator according to a first embodiment of the present invention.

FIG. 2 is an internal circuit diagram conceptually showing the internal structure of the oxygen concentrator of FIG. 1.

FIG. 3 is a plan view of the oxygen concentrator of FIG. 1.

FIG. 4 is a right side view of the oxygen concentrator of FIG. 1.

FIG. 5 is a left side view of the oxygen concentrator of FIG. 1.

FIG. 6 is a back view of the oxygen concentrator of FIG. 1.

FIG. 7 is a bottom view of the oxygen concentrator of FIG. 1.

FIG. 8 is a partial sectional side view of the oxygen concentrator of FIG. 1.

FIG. 9A is an explanatory view schematically showing the sectional structure of the oxygen concentrator of FIG. 1.

FIG. 9B is an explanatory view schematically showing the sectional structure of comparative example 1.

FIG. 9C is an explanatory view schematically showing the sectional structure of comparative example 2.

### DESCRIPTION OF REFERENCE NUMERALS

Reference numerals used to identify various features in the drawings include the following.

1: oxygen concentrator

5: display

50: housing

51: outer circumferential surface portion

52: front face portion

54: bottom face portion

56: top face portion

60, 70: side face portion

64, 74: extension portion

80: back face portion

82: handle portion

84: back face main body portion

91: first opening

92: second opening

94: through hole

F: imaginary plane

### DETAILED DESCRIPTION OF THE PREFERRED EMBODIMENTS

The invention will now be described in further detail with reference to the drawings. However, the present invention should not be construed as being limited thereto.

### First Embodiment

### 1. Outline of Oxygen Concentrator

First, the outline of an oxygen concentrator 1 will be described.

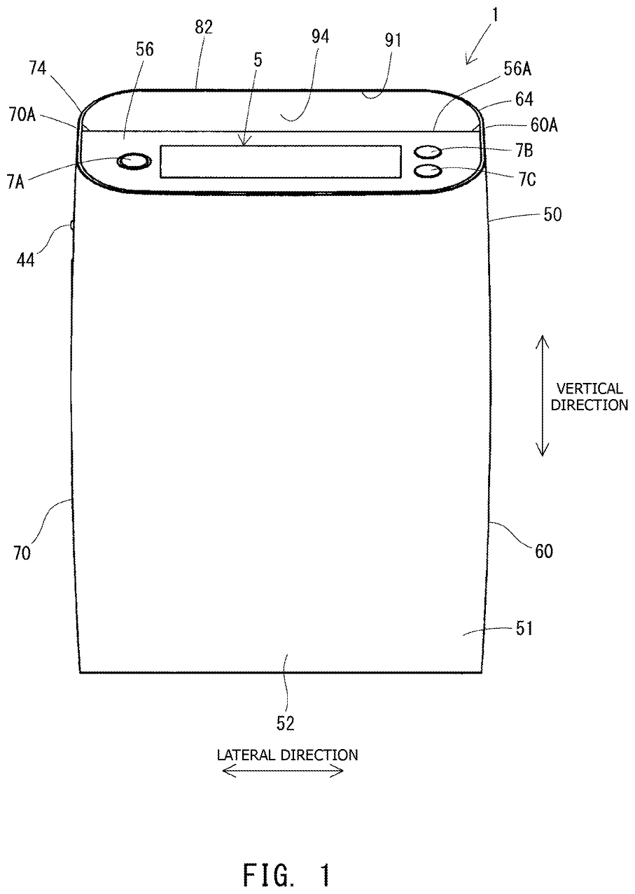

The oxygen concentrator 1 shown in FIG. 1 is adapted to supply oxygen mainly to patients with respiratory diseases, etc., and is used with a publicly known cannula connected to a cannula connection 44 (oxygen-concentrated gas outlet).

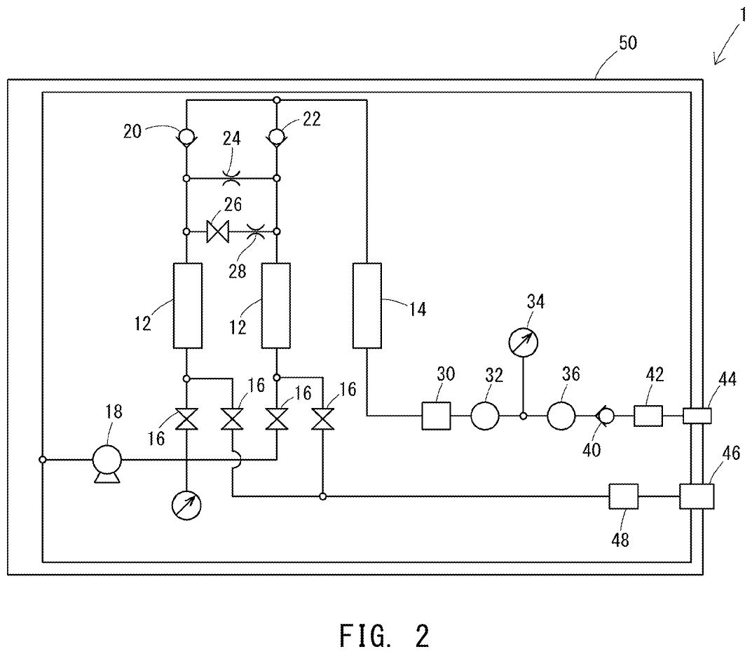

As shown in FIG. 2, the oxygen concentrator 1 functions as, for example, a portable oxygen concentrator, and includes a plurality of components such as adsorber columns 12 containing an adsorbent capable of selectively adsorbing nitrogen contained in material air, a storage tank 14 for temporarily storing oxygen-concentrated gas produced in the adsorber columns 12, a gas transfer device housing box for housing a gas transfer device 18 for transferring the material air, solenoid valves 16 for opening/closing gas passages connected to the adsorber columns 12, and a control unit (not shown) for controlling the gas transfer device 18 and the solenoid valves 16. These components are accommodated in a housing 50 and supported by supports (frames, etc.) fixedly accommodated in the housing 50.

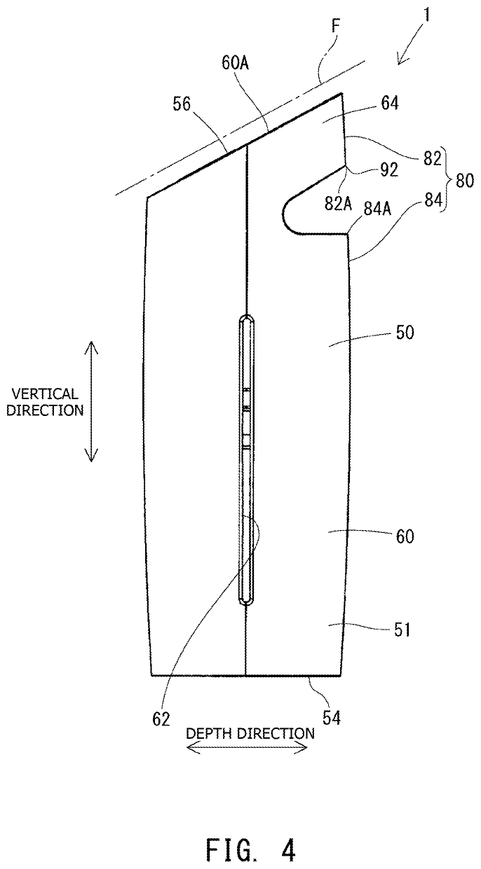

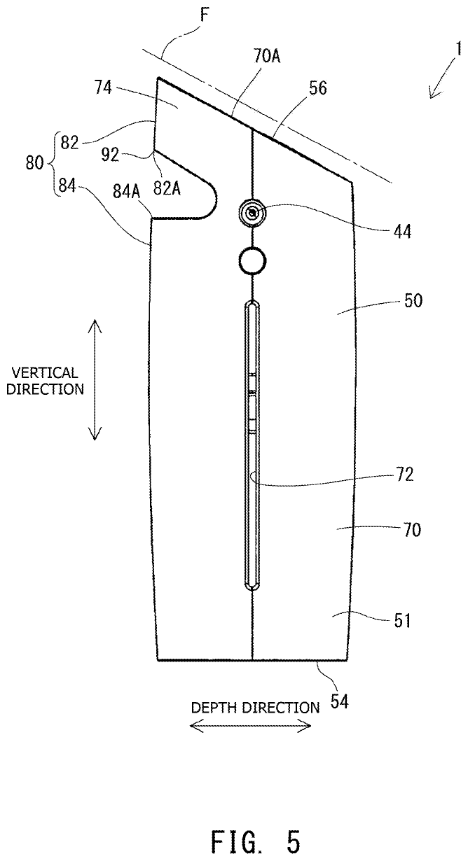

The oxygen concentrator 1 is of a pressure swing adsorption type in which oxygen-concentrated gas is produced by swinging the pressure of the adsorber columns 12 containing the adsorbent for selectively adsorbing nitrogen contained in ambient air (material air). In the oxygen concentrator 1, the material air is taken in from intake/exhaust ports 62 and 72 (FIGS. 4 and 5) through unillustrated filters and is then transferred under pressure to the adsorber columns 12. When the internal pressure of the adsorber columns 12 increases as a result of the material air being transferred under pressure to the primary side (gas inlet side) of the adsorber columns 12, nitrogen contained in the material air is adsorbed by the adsorbent. As a result, oxygen-concentrated gas having high oxygen concentration is discharged from the secondary side (outlet side) of the adsorber columns 12 (adsorption process). The thus-produced oxygen-concentrated gas is temporarily stored in the storage tank 14 and is then taken out as needed from the cannula connection 44 (oxygen-concentrated gas outlet).

Gas remaining in the adsorber columns 12 which have undergone the above adsorption process is discharged as exhaust gas from an exhaust gas outlet 46 (silencer). On this occasion, the pressure of the adsorber columns 12 drops. As a result, nitrogen is desorbed from the adsorbent, thereby restoring the nitrogen adsorbing capability of the adsorbent (restoration process). Nitrogen desorbed from the adsorbent is discharged as exhaust gas. Accordingly, exhaust gas discharged from the exhaust gas outlet 46 is nitrogen-enriched gas having a high nitrogen concentration.

Reference numerals 20 and 22 denote check valves; reference numerals 24 and 28 denote orifices; and reference numeral 26 denotes an equalizer valve. Reference numeral 30 denotes a flow rate controller; reference numeral 32 denotes an oxygen concentration detector; and reference numeral 34 denotes a pressure detector. Reference numeral 36 denotes a flow rate detector, and reference numeral 40 denotes a check valve. Reference numeral 42 denotes a humidifier. Reference numeral 48 denotes a silencer tank. Since these components and other components of the oxygen concentrator 1 are similar to those of a conventional pressure swing adsorption type oxygen concentrator, the description of specific functions and operations thereof is omitted.

### 2. Structure of Housing

Next, the structure of the housing 50 will be described.

The oxygen concentrator 1 has a housing 50 for accommodating the above-mentioned plurality of components (see FIG. 2), as well as a display 5 exposed from the surface of the housing 50.

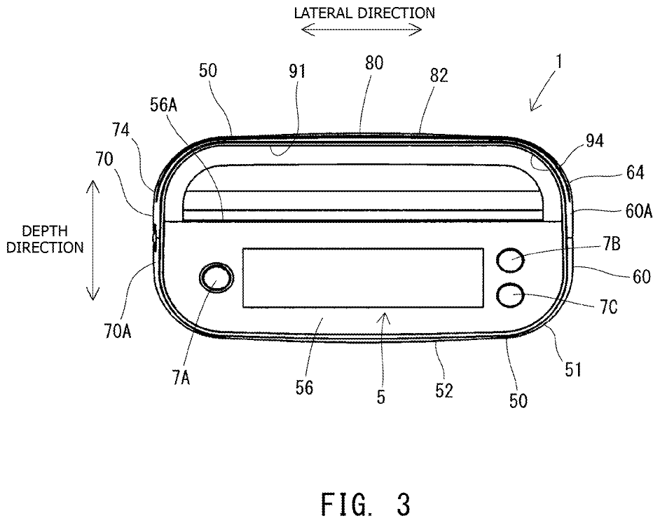

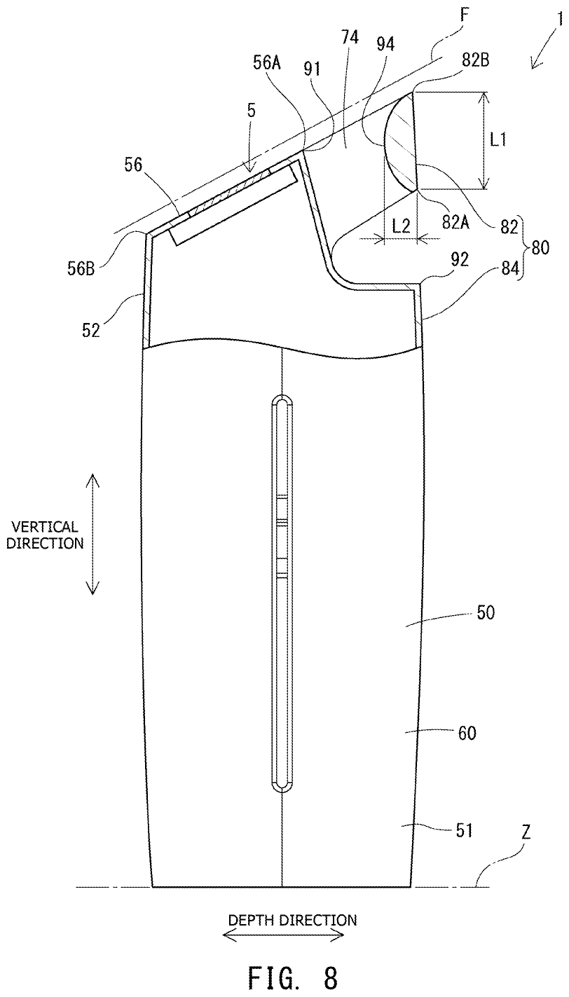

As shown in FIG. 3, the housing 50 includes a front face portion 52 and a back face portion 80 disposed in a mutually facing manner with respect to a predetermined depth direction, and a pair of side face portions 60 and 70 disposed in a mutually facing manner with respect to a predetermined lateral direction orthogonal to the depth direction. The front face portion 52, the back face portion 80, and the two side face portions 60 and 70 constitute an outer circumferential surface portion 51. According to the present structure, the depth direction is a direction in which the front face portion 52 and the back face portion 80 face each other, and the lateral direction is a direction in which the two side face portions 60 and 70 face each other. A direction orthogonal to the depth direction and to the lateral direction is taken as a vertical direction. Specifically, when the oxygen concentrator 1 is placed on an imaginary plane (e.g., a plane Z shown in FIG. 8), a direction orthogonal to the plane is the vertical direction, and, in the plan view of FIG. 3 (a view of the oxygen concentrator I placed on the plane Z of FIG. 8 viewed in the vertical direction from a side opposite the plane Z), the lateral direction is the longer-side direction of the housing 50, while the depth direction is the shorter-side direction of the housing 50. In the present specification, the vertical direction is an example of the “axial direction,” and an upper side with respect to the vertical direction (a side where a top face portion 56 is present) corresponds to one side in the axial direction, while a lower side with respect to the vertical direction (a side where a bottom face portion 54 is present) corresponds to the other side in the axial direction. The front face portion 52 extends along the vertical direction (axial direction), and the back face portion 80 faces the front face portion 52. The two side face portions 60 and 70 are disposed between the front face portion 52 and the back face portion 80 while facing each other.

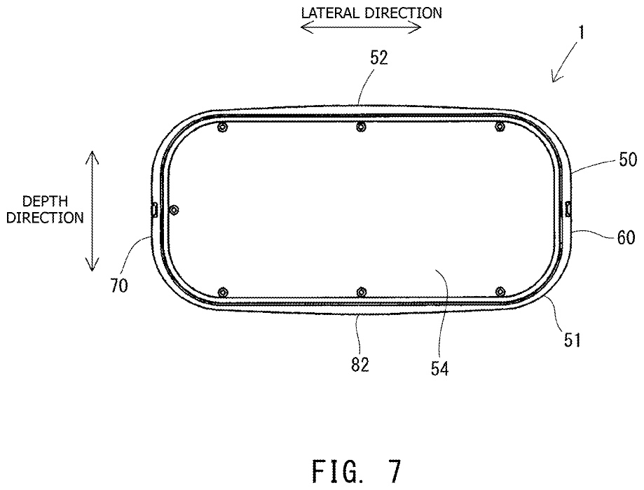

As shown in FIG. 4, the bottom face portion 54 is attached to the lower end of the outer circumferential surface portion 51 so as to close a lower end opening of the outer circumferential surface portion 51 and to face a lower side (the other side in the axial direction) (see also FIG. 7). Further, the top face portion 56 is attached to an upper end of the outer circumferential surface portion 51 so as to partially close an upper end opening of the outer circumferential surface portion 51 (see also FIG. 3). The housing 50 is a box whose interior space is enclosed by the outer circumferential surface portion 51, the bottom face portion 54, and the top face portion 56.

As shown in FIGS. 4 and 5, the top face portion 56 is inclined along a predetermined imaginary plane F extending in parallel with the lateral direction and increasing in level toward the back side (inclined from the front face portion 52 toward the one side in the axial direction as viewed in the direction from the front face portion 52 toward the back face portion 80), and faces an upper side (the one side in the axial direction). As shown in FIG. 3, a back-side end 56A of the top face portion 56 is disposed (offset) frontward of the back face portion 80. In the example of FIGS. 4 and 5, the surface of the top face portion 56 is disposed approximately in parallel with the imaginary plane F, and the surface of the display 5 is also disposed approximately in parallel with the imaginary plane F. The imaginary plane F is inclined at a predetermined angle (e.g., 10° to 80°, desirably 20° to 70°) with respect to the depth direction.

As shown in FIGS. 4 and 5, upper end portions 60A and 70A of the two side face portions 60 and 70, respectively, are inclined along the top face portion 56 so as to increase in level toward the back side. As shown in FIG. 4, an upper end portion of the side face portion 60, including the upper end 60A thereof, has an extension portion 64 which extends backward of the back-side end 56A of the top face portion 56 along the imaginary plane F. As shown in FIG. 5, an upper end portion of the side face portion 70, including the upper end 70A thereof, has an extension portion 74 which extends backward of the back-side end 56A of the top face portion 56 along the imaginary plane F. A part of the upper end portion 60A (end portion on the one side in the axial direction), which part is located forward of the extension portion 64, and a part of the upper end portion 70A (end portion on the one side in the axial direction), which part is located forward of the extension portion 74, are inclined portions extending along the top face portion 56 from the front face portion 52 toward the back face portion 80.

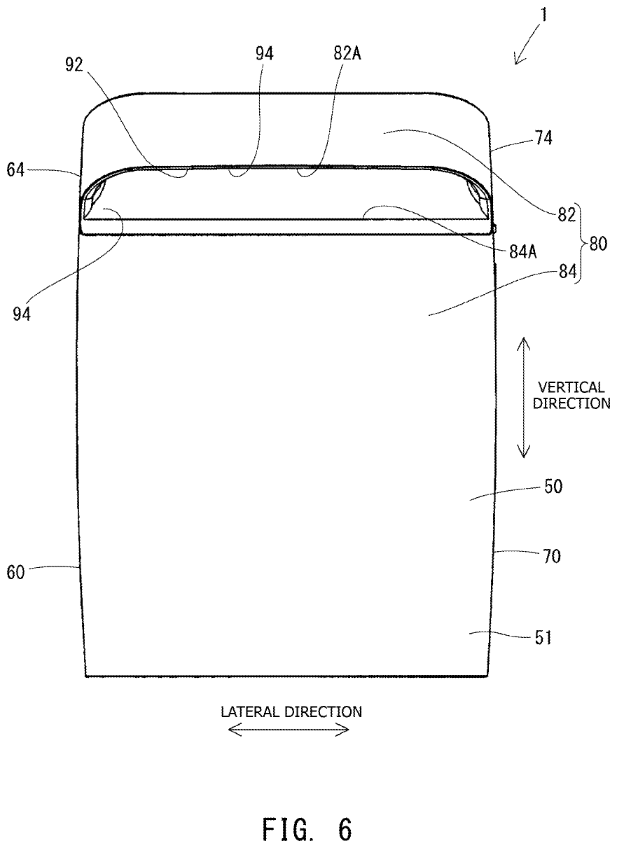

As shown in FIG. 6, the back face portion 80 has a handle portion 82 extending between the extension portions 64 and 74 of the two side face portions 60 and 70 in a bridging manner, and a back face main body portion 84 located downward of the handle portion 82 and facing the handle portion 82 in the axial direction with a gap intervening therebetween. As shown in FIG. 8, the handle portion 82 is such that the maximal width LI along the vertical direction is greater than the maximal width L2 along the depth direction. Also, an upper end 82B of the handle portion 82 is disposed at a position higher than the position of the back-side end 56A (upper end) of the top face portion 56, and a lower end 82A of the handle portion 82 is disposed at a position lower than the position of the back-side end 56A (upper end) of the top face portion 56.

As shown in FIGS. 1 and 3, in the housing 50, a first opening 91 is defined at least by the back-side end 56A of the top face portion 56, the two extension portions 64 and 74, and the handle portion 82. Further, as shown in FIGS. 4 to 6, a second opening 92 is defined at least by the lower end 82A of the handle portion 82 and an upper end 84A of the back face main body portion 84. The second opening 92 is disposed below the first opening 91. The upper end of the second opening 92 corresponds to the lower end of the handle portion 82 and is disposed at a position lower than the back-side end 56A (upper end) of the top face portion 56. The lower end of the second opening 92 corresponds to the upper end 84A of the back face main body portion 84 and is disposed at a position lower than a front end 56B (lower end) of the top face portion 56.

As shown in FIG. 8, a through hole 94 is formed such that the through hole 94 extends downward from the first opening 91 and has an interior space continuously extending between the first opening 91 and the second opening 92. The through hole 94 is a laterally wide hole and allows a user to insert his/her hand thereinto for holding the handle portion 82.

The display 5 is exposed from the top face portion 56 and has a laterally elongated shape. The upper surface (exposed surface) of the display 5 is disposed approximately in parallel with the above-mentioned imaginary plane F. The display 5 is, for example, a liquid crystal display device and can display various kinds of information. The display 5 has a laterally elongated rectangular shape and is disposed in a laterally central region of the top face portion 56. In the top face portion 56, a power button 7A is disposed on one side in the lateral direction (left side) of the display 5, and flow control buttons 7B and 7C are disposed on the other side in the lateral direction (right side) of the display 5.

### 3. Effect

In the oxygen concentrator 1, portions (extension portions 64 and 74) of the two side face portions 60 and 70 are disposed so as to extend toward the back face portion 80 and thereby utilize a space located backward of the back-side end (end toward the back face portion 80) of the top face portion 56, and a portion (handle portion 82) of the back face portion 80 is connected to the two extension portions 64 and 74. By virtue of such a structure, the user can hold the portion of the back face portion 80 as a handle. Further, since the handle portion 82, and the two extension portions 64 and 74 connected to the opposite sides of the handle portion 82 are less prone to cause an increase in the dimension in the axial direction, an increase in the overall size of the oxygen concentrator 1 in the axial direction can be suppressed. That is, in the form of use of the oxygen concentrator 1 with the one side in the axial direction corresponding to the upper side thereof and the other side in the axial direction corresponding to the lower side thereof, an increase in the overall vertical size of the oxygen concentrate can be suppressed.

Specifically, the oxygen concentrator 1 has the top face portion 56 provided along the predetermined imaginary plane F sloping frontward (a predetermined imaginary plane extending in parallel with the lateral direction and inclined so as to increase in level toward the back side), and the two extension portions 64 and 74 including the upper ends of the two side face portions 60 and 70 and disposed along the imaginary plane F. The handle portion 82 is connected to the two extension portions 64 and 74 in a bridging manner By virtue of such a structure, the handle portion 82 and the two extension portions 64 and 74 connected to the opposite ends of the handle portion 82 do not cause an increase in vertical dimension, whereby an increase in the overall vertical size of the oxygen concentrator 1 can be suppressed.

Further, the through hole 94 adjacent to the handle portion 82 is formed so as to extend downward from the first opening 91 (an opening defined at least by the back-side end 56A (an end toward the back face portion 80) of the top face portion 56, the two extension portions 64 and 74, and the handle portion 82 of the housing 50) and has an interior space continuously extending between the first opening 91 and the second opening 92 (an opening defined at least by the lower end (an end 82A on the other side in the axial direction) of the handle portion 82 and the upper end (an end 84A on the one side in the axial direction) of the back face main body portion 84 and disposed downward of the first opening 91). Thus, the interior space of the through hole 94 allows the user to insert his/her hand therein for holding the handle portion 82. Further, since the through hole 94 extends vertically and is adjacent to the handle portion 82 located backward thereof, despite imparting a large vertical thickness to the handle portion 82, the overall vertical size of the oxygen concentrator 1 is not prone to increase. Therefore, the strength of the handle portion 82 can be readily ensured while an increase in the overall vertical size of the oxygen concentrator 1 is suppressed.

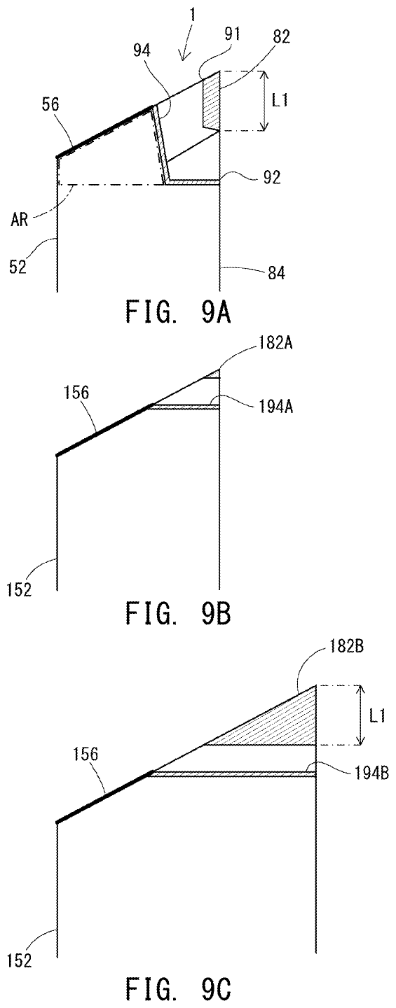

FIGS. 9A to 9C specifically show the effect of the structure of the first embodiment. FIG. 9A is an explanatory view schematically showing the structure of FIG. 8. FIG. 9A shows the through hole 94 and its periphery in section. FIG. 9B shows an oxygen concentrator which has the same outline (size) as that of the oxygen concentrator 1 and which includes, in place of the through hole 94 shown in FIG. 9A, a through hole 194A extending horizontally in the depth direction from the back-side end of a top face portion 156 of the oxygen concentrator. Notably, the oxygen concentrator of FIG. 9B is such that a front face portion 152 has the same shape as that of the front face portion 52 of the oxygen concentrator 1 shown in FIG. 9A. Also, a top face portion 156 has the same shape as that of the top face portion 56 of the oxygen concentrator 1 shown in FIG. 9A. In the oxygen concentrator of FIG. 9B which has the same size as that of the oxygen concentrator 1 while having the through hole 194A extending horizontally in the depth direction, a handle portion 182A has a reduced vertical width and width along the depth direction. As a result, lack of strength of the handle portion 182A is a concern. Further, the size of opening of the through hole 194A is reduced. In contrast, the oxygen concentrator 1 of the first embodiment shown in FIG. 9A solves such problems.

FIG. 9C shows an oxygen concentrator whose front face portion 152 and top face portion 156 have the same shapes as those of FIG. 9B (i.e., the same shapes as those of FIG. 9A) while having a through hole 194B extending horizontally in the depth direction and whose size is increased so as to impart the same vertical width L1 of the oxygen concentrator 1 of FIG. 9A to a handle portion 182B. The oxygen concentrator of FIG. 9C has an increased depth and height, unavoidably resulting in an increase in size and weight. Further, in the structure of FIG. 9C, the vertical width of opening of the through hole 194B is small. In order to impart approximately the same opening size as that of FIG. 9A to the through hole 194B, the vertical size of the oxygen concentrator must be further increased. As compared with the structure of FIG. 9C, the oxygen concentrator 1 of the present embodiment advantageously has a reduced size and weight.

In the oxygen concentrator 1, the handle portion 82 is such that a maximal width along the vertical direction (axial direction) is greater than a maximal width along the depth direction (direction from the front face portion 52 to the back face portion 80). By employing such a dimensional feature, since a greater vertical width can be imparted to the handle portion 82, when the user lifts the oxygen concentrator 1 while holding the handle portion 82, an upward deflection of the handle portion 82 is readily restrained. Consequently, the strength of the handle portion 82 is further ensured. Further, despite such a relative increase in the vertical maximal width of the handle portion 82, the overall vertical size of the oxygen concentrator 1 is not prone to increase.

In the oxygen concentrator 1, the display 5 is exposed from the top face portion 56. This implements an oxygen concentrator allowing easy visual recognition of the display 5 from the front side and the upper side. Further, since the display 5 is exposed from the top face portion 56 sloping down forward, a space (denoted by reference mark AR in FIG. 9A) under the inclined face portion (top face portion 56), which encounters difficulty in disposing large components, can be effectively utilized for disposing display-related components (display panel, circuit board, wiring, etc.).

### Other Embodiments

The present invention is not limited to the above embodiment and its modifications, but may be embodied in various other forms without departing from the spirit of the invention. For example, in order to solve, partially or entirely, the above-mentioned problem or yield, partially or entirely, the above-mentioned effects, technical features of the embodiment and modifications corresponding to technical features of the modes described above can be replaced or combined as appropriate. Also, the technical feature(s) may be eliminated as appropriate unless the present specification mentions that a certain technical feature is mandatory. Modified embodiments, for example, include the following.

In the above embodiment, the handle portion is such that the maximal vertical width is greater than the maximal width along the depth direction. However, the vertical maximal width may be approximately equal to the maximal width along the depth direction, or the vertical maximal width may be smaller than the maximal width along the depth direction.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

This application is based on Japanese Patent Application No. JP 2018-093536 filed May 15, 2018, the disclosure of which is incorporated herein by reference in its entirety.

What is claimed is:

1. An oxygen concentrator comprising a housing accommodating a plurality of components and extending in an axial direction, wherein the housing comprises
an outer circumferential surface portion having a front face portion extending along the axial direction, a back face portion disposed opposite the front face portion, and a pair of side face portions disposed between the front face portion and the back face portion in a mutually facing manner,
a top face portion facing toward one side in the axial direction, and
a bottom face portion facing toward the other side in the axial direction;
the top face portion is disposed so as to be inclined toward the one side in the axial direction in a direction from the front face portion toward the back face portion and such that an end of the top face portion located toward the back face portion is offset from the back face portion toward the front face portion;
an end portion of each of the two side face portions, which end portion is located on the one side in the axial direction, has an inclined portion extending from the front face portion toward the back face portion along the top face portion, and an extension portion extending toward the back face portion further than the top face portion;
the back face portion has a handle portion extending between the extension portions of the two side face portions in a bridging manner, and a back face main portion facing the handle portion in the axial direction with a gap intervening therebetween; and
a first opening is defined at least by the end of the top face portion on the side toward the back face portion, the two extension portions and the handle portion, a second opening is defined at least by an end of the handle portion on the other side in the axial direction and an end of the back face main portion on the one side in the axial direction and is disposed on the other side in the axial direction with respect to the first opening, and a through hole is formed which extends from the first opening toward the other side in the axial direction and has an interior space continuously extending between the first opening and the second opening.

2. The oxygen concentrator as claimed in claim 1, wherein a maximal width of the handle portion along the axial direction is greater than a maximal width along a direction from the front face portion to the back face portion.

3. The oxygen concentrator as claimed in claim 1, further comprising a display provided on the top face portion.
